(19) United States
(12) Patent Application Publication
(10) Pub. No.: US 2010/0198322 A1
(43) Pub. Date: Aug. 5, 2010

(54) PERSONAL TEMPERATURE REGULATOR

(75) Inventors: DANIEL M. JOSEPH, LOS ANGELES, CA (US); MARK A. REICHOW, VALENCIA, CA (US)

Correspondence Address: DISNEY ENTERPRISES, INC.;c/o Marsh Fischmann & Breyfogle LLP, 8055 East Tufts Avenue, Suite 450, Denver, CO, 80237, US

(73) Assignee: DISNEY ENTERPRISES, INC., BURBANK, CA (US)

(21) Appl. No.: 12/366,339

(22) Filed: Feb. 5, 2009

(51) Int. Cl. A61F 7/00

(52) U.S. Cl. 607/108

(57) ABSTRACT

An apparatus for regulating a user's core body temperature. The apparatus includes a thermoelectric module with a Peltier unit with a heat transfer surface at a temperature differing from ambient. The apparatus includes a thermally conductive member with a first side abutting the surface of the Peltier unit and with a second side for contacting the user's skin when the apparatus is worm. The thermally conductive member is formed of a flexible and conformable material such that the second side conforms to the topography of the skin when pressed against the user's body. The conformable material may be a thermally conductive elastomeric material or flexible polymer material. Use of a conformable material to contact the user's skin allows an effective heat conduction pathway to be formed between the thermally conductive member and the user's skin, e.g., more than half of the available surface area may solidly contact the skin.

### BACKGROUND OF THE INVENTION

1. Field of the Invention

The present invention relates, in general, to cooling devices for individual use such as devices that can be worn or placed in contact with the skin, and, more particularly, to a portable or personal temperature regulator device that uses a thermal electric module (TEM) or a thermal-voltaic module to transfer heat away from a thermally conductive element positioned in contact with the user's skin.

2. Relevant Background

There are many situations in which it is desirable for a person to be able to control or regulate their temperature. For example, many work environments involve a worker being exposed to relatively low or high temperatures. In some cases, the temperature variances can be addressed simply by wearing warmer or cooler clothing. However, protective clothing, uniforms, and other clothing worn by a worker or employee may contribute to the problem by trapping heat that raises the worker's body temperature. For example, a worker that is required to wear a uniform such as a costume at a theme park or anti-contamination coveralls at an environmental remediation site, both of which may experience temperatures in the 80 to 100° F. range or higher, will experience their core body temperature quickly rising to uncomfortable levels. Additionally, many outdoor entertainment facilities such as theme parks and sports stadiums face problems with keeping their visitors comfortable (e.g., not too hot or too cold), with heat exhaustion and discomfort often leading to shorter visits, less enjoyable experiences, or fewer ticket sales.

When cooling is the main need, a basic solution is to provide fans that move the air and provide some evaporative cooling, and individuals may carry portable electric fans or use hand fans to provide some personal control over cooling. Evaporative cooling is enhanced by moistening the skin with water rather than relying only on an individual's perspiration to provide a liquid to evaporate. For example, portable misting devices are used worldwide to allow individuals to cool themselves while attending sporting event and other outdoor activities such as amusement parks. In general, these devices make use of evaporative cooling and provide a mist of water on a person's skin that is then evaporated by air flowing from a fan (e.g., provide water-activated types of evaporative cooling that are typically very inefficient). In other words, existing misting devices typically provide a combination of a battery operated fan to provide a flow of air and a pump adapted to provide an atomized mist spray of water. Other evaporative cooling designs are also used, but each of these designs requires that a person spray liquid on their skin and that the skin is exposed to allow evaporation. Many people do not like being sprayed with water or having to carry around heavy containers. Also, such evaporative cooling is not particularly useful for workers that have to cover much if not all of their skin, and these temperature regulation devices are only useful in cooling and not in situations where it desirable to warm a person.

More recently, there have been attempts to use thermoelectric devices to provide personal cooling and/or heating. Thermoelectric devices, which may also be called Peltier devices, TEM devices, thermo-voltaic modules, thermoelectric cooler, and so on, use the Peltier effect to create a heat flux between the junction of two different types of materials. A Peltier-based device is a solid-state active heat pump that transfers heat from one side of the device to the other against a temperature gradient (e.g., from cold to hot) when powered by an electric source (e.g., with consumption of electrical energy). Simply connecting a thermoelectric device to a DC voltage such as a battery or other power source will cause one side to cool while the other side of the device to warm.

To date, thermoelectric devices have not been widely adopted for personal temperature control or regulation. One of the problems with previously proposed designs is that Peltier devices required relatively large voltages and high currents to be useful, and even at higher voltages and currents (such as 15 VDC and up to 5 amps or the like) the devices often could only provide a several degree temperature reduction (e.g., 4 to 8° F.), while 10 to 40° F. differential is often needed to provide adequate cooling of a person's skin. Some lower voltage Peltier devices have been developed that can create larger temperature differences relative to ambient, but these devices still have not been widely adopted for personal use. Another problem with prior devices is that heat transfer to the Peltier device has been inefficient and/or uncomfortable for the user.

For example, some proposed devices have used a metal plate as a heat transfer element. The metal plate has one side mated to the Peltier device and a second side positioned near the body of the user (such as against the user's skin) to attempt to remove heat from the user's body to the Peltier device. One design called for the metal heat transfer element to be provided in a heat band, but the rigidness of the element made the device uncomfortable to wear and also provided limited heat transfer surface area. For example, the rigidness of the metal caused the band to only contact the user's head at “high” points or areas, which significantly reduces the heat transfer surface area and also creates pressure points that leads to user discomfort. As a result, such designs were limited to use with smooth and/or flat portions of the user's body to try to increase the area in contact with the skin. Pads may be provided between the heat transfer element and the user's skin to increase comfort, but such a pad lowers the heat transfer rate from the skin to the heat transfer element.

Other devices have been proposed that utilize metallic mesh as the heat transfer element, but such a design significantly reduces the heat transfer surface area as much of the element is a void or air gap (e.g., a chain-link fence is mostly air spaces or gaps between the wires). Also, the use of mesh typically requires a pocket or support structure to contain the mesh element. However, a pocket or similar holder for the metallic mesh heat transfer element places a thermal insulator or lower thermal conductivity material (such as a cloth or other fabric) between the heat transfer element and the user's skin, which reduces heat transfer effectiveness to undesirably low levels.

Hence, there remains a need for an improved design for a device that utilizes a thermoelectric device to allow an individual to regulate their temperature. Preferably such a device would allow the user to lower or raise their core body temperature while being relatively comfortable to use and lightweight.

### SUMMARY OF THE INVENTION

The present invention addresses the above problems by providing a personal temperature regulator that allows a user to cool (or heat) their core body temperature. The personal temperature regulators include thermoelectric devices that utilize the Peltier effect (which may be called Peltier units or devices) to create a temperature gradient between two differing materials when powered with electrical current. The thermoelectric device can be powered by a low voltage power source such as a rechargeable battery but yet can achieve a temperature at a heat transfer surface of up to 30 to 40 F or more. To provide cooling to the user, a thermally conductive member is positioned in contact with the heat transfer surface of the thermoelectric device. The personal temperature regulator is adapted to be worn by the user, and the opposite side of the thermally conductive member is positioned against the user's skin to transfer heat away (or toward) to cool (or heat) the user's skin and blood flowing below the skin surface.

Significantly, the thermally conductive member is formed of a flexible and conformable material such as a thermally conductive polymer or elastomer, and, when pressed against the skin by a collar, band, strap, or the like, the conformable material conforms to the topography or shape of the user's skin to achieve a relatively large heat transfer surface (e.g., up to 50 to 75 percent or more of the contact surface of the thermally conductive member may be in solid or direct contact with the user's skin). The flexible and conformable material allows the personal temperature regulator to be worn about a user's neck, wrists, knees, head, lower back, and other areas of the body that may have relatively irregular surfaces and typically have more arteries/veins near the skin's surface, which facilitates better regulation of a user's core body temperature by transferring heat away (or to) the user's flowing blood.

More particularly, an apparatus is provided for regulating the core body temperature of a user or person wearing the apparatus. The apparatus includes a thermoelectric module with a Peltier unit that provides a heat transfer surface at a temperature offset or differing from the ambient temperature (e.g., 30 to 40° F. below (or above) the ambient temperature around the user). The apparatus also includes a thermally conductive member with a first side abutting the heat transfer surface of the Peltier unit and with a second side for contacting an area of skin of the user's body when the apparatus is worn by the user (e.g., on the user's neck, head, wrist, knee, or the like). The thermally conductive member is formed of a flexible and conformable material such that the second side conforms to the topography of the skin when it is pressed against the user's body, and, in one embodiment, the conformable material is a thermally conductive elastomeric material (e.g., an elastomer with a thermal conductivity greater than about 1 W/mK and a hardness of less than about Shore A 40).

Use of a conformable material that contacts the user's skin allows a heat conduction pathway to be formed between the thermally conductive member and the user's skin with up to 50 to 75 percent or more of the second side or skin-contacting side of the thermally conductive member (rather than a relatively ineffective contact fraction achievable with a rigid element). The thermoelectric module may include a housing supporting the Peltier unit and also a low voltage power source (e.g., a 9 Volt rechargeable battery or lower voltage source) to provide an electrical current to the Peltier unit to achieve the temperature of the heat transfer surface. A positioning element such as a band, strap, collar, or the like may extend outward from the housing to at least partially cover the thermally conductive member and, when worn by the user, to apply a compressive force against the first side of the member to cause the second side to conform to the user's skin.

### BRIEF DESCRIPTION OF THE DRAWINGS

FIGS. 1 and 2 illustrate perspective views of a personal temperature regulator (e.g., personal cooling/heating device or assembly) as it may appear when worn or positioned on a user's neck;

FIG. 3 illustrates a perspective view of a personal temperature regulator similar to that shown in FIGS. 1 and 2 that is adapted for cooling/heating a user when worn on a wrist;

FIG. 4 is a functional block diagram of a personal temperature regulator in accordance with an embodiment of the invention (e.g., as may be used to implement the devices shown in FIGS. 1-3);

FIG. 5 illustrates a sectional view of the personal temperature regulator of FIGS. 1 and 2 showing components of the heat transfer module and, significantly, showing the thermally conductive member molding or conforming to the irregular surface presented by the user's skin on their neck (e.g., a person's neck is not a smooth cylindrical surface) to achieve a large heat transfer surface or contact surface;

FIG. 6 illustrates use of a rigid metallic plate for a heat transfer element as attempted in some prior thermoelectric cooling devices showing ineffective heat transfer surface or contact that is achieved;

FIGS. 7A and 7B illustrate another embodiment of a personal temperature regulator utilizing a thermally conductive assembly with a number of flexible and/or resiliently deformable (“conformable”) members used to cool a user's neck, upper/mid back, and lower back;

FIG. 8 illustrates another embodiment of a personal temperature regulator using a thermally conductive assembly adapted to cool portions of a user's head and also including additional regulator subassemblies to cool the user's legs near the knees;

FIGS. 9-12 illustrate views of another embodiment of a personal temperature regulator similar to that shown in FIGS. 1 and 2 using a more rigid collar linked to a housing for the heat transfer or thermoelectric cooling/heating module and showing other features useful in some implementations including a deformable or “squishy” interface on a user's skin; and

FIGS. 13 and 14 illustrate a costume (or uniform, protective clothing, or the like) including headgear with a personal temperature regulator in accordance with another embodiment of the invention.

### DETAILED DESCRIPTION OF THE PREFERRED EMBODIMENTS

Briefly, embodiments of the present invention are directed to personal temperature regulators (e.g., personal cooling products). The temperature regulators make use of flexible and conformable thermally conductive elements/members (e.g., sheets of thermally conductive polymers, elastomers, and the like) to provide significantly large heat transfer surfaces/contact areas with a user's skin. The temperature regulators may have many applications, but one of the problems that faces outdoor entertainment facilities such as theme parks, sports stadiums, and the like is heat related exhaustion and discomfort due, such as when temperatures are 85 to 100° F. or higher. The temperature regulators are designed to directly contact a user's skin and bring the user's core body temperatures down to a more comfortable temperature range. Briefly, the regulators include a thermoelectric device (e.g., a Peltier device) that acts to remove from the thermally conductive member when coupled to a rechargeable or other battery (or other source of electrical current). A heat sink and fan combination, in turn, remove heat from the thermoelectric device.

Embodiments of the temperature regulator are adapted to be worn by a user with the thermally conductive member positioned in contact with the user's skin. For example, embodiments of the regulator may be designed for wearing on or near the user's neck, with an adjustable band, strap, collar, or the like being used to position the thermally conductive member against the user's skin and, typically, to apply a force upon the member to urge a heat transfer/contact surface or side into contact with the skin. A person's skin is typically not perfectly smooth and may have contours and recessed surfaces especially near protruding bone or structural areas such as near the back of the neck, the front of the neck, the spine, the forehead/temple, and so on, and the use of a thermally conductive member that is formed from a conformable or compressible (e.g., resiliently deformable) material such as certain polymers or an elastomeric material allows the thermally conductive material to take the shape of the user's skin, even if it is irregular and/or non-planar, such that a relatively large portion of the member is in heat transfer or direct contact with the user's skin (e.g., typically at least 50 percent of the member's heat transfer/user contact surface is in contact with a user's skin while some embodiments are able to achieve 75 to 90 percent or more contact).

This large area of contact between the skin and the thermally conductive member increases heat transfer effectiveness of the regulator. The personal temperature regulator may be particularly effective when the thermally conductive member is positioned on the user in an area where blood flow is prevalent such as near the neck, the wrists, the inner surfaces of the knees, and so on where arteries and veins are near the skin's surface. For example, the thermally conductive member may be provided in a neck collar, a wrist band, a knee pad/band, a hat/helmet, a vest, coveralls, a full body costume and/or applied to the skin in other ways such as with straps that force the conformable material against the spine and/or lower back or other portions of the body. Typically, though, the conductive member has a contact side or surface that is placed in direct contact with the skin (e.g., a pocket or the like is not used unless it is formed so as to support the member without positioning an insulating layer of fabric or cloth between the skin and the thermally conductive member). In some embodiments, the personal temperature regulator is rechargeable (e.g., uses rechargeable batteries as a power source for the fan, Peltier, and any control components/circuitry). The regulator product is also ergonomically designed to provide comfortable use and to position flexible components such as the thermally conductive member and strap/band used to apply a conforming pressure/force in areas requiring flexibility (e.g., against the inner surfaces of the knee). The cooling effect is significantly greater than prior cooling devices making use of Peltier devices, with some embodiments being able to achieve skin to ambient temperature differences of up to about 30 to 40 degrees or more (e.g., when it is 110° F. ambient temperature the skin temperature can be lowered to about 80 to 70° F. or the like). These and other advantages will become clear from the following description.

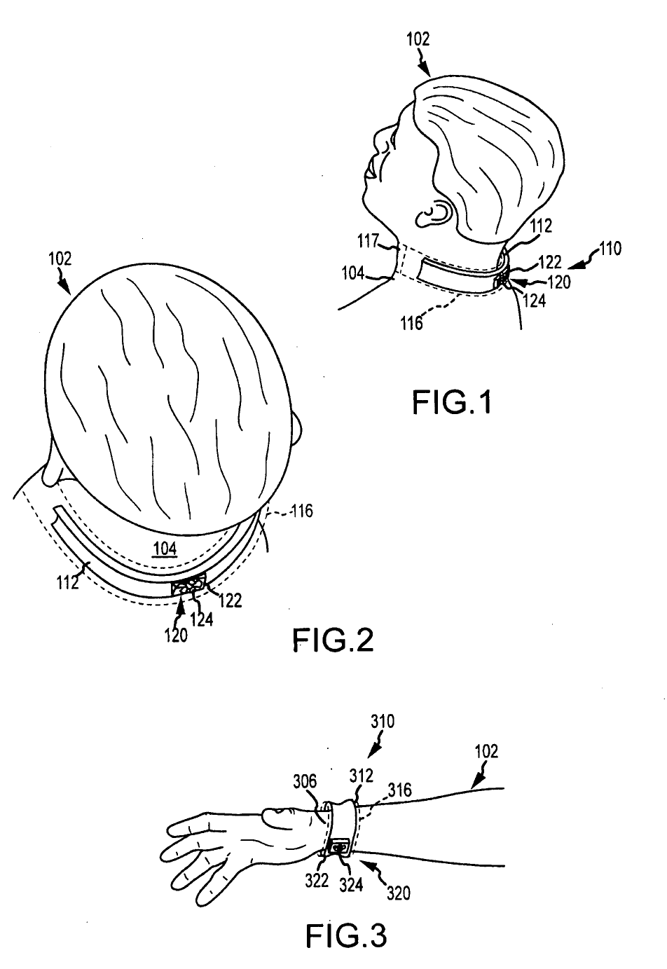

FIGS. 1 and 2 illustrate a personal temperature regulator (or personal cooling/heating assembly) 110 in accordance with an embodiment of the invention. As discussed above, it is often desirable to better control a person's core body temperature by locating the heat transfer components (e.g., the thermally conductive member) not only against the person's skin but against the skin in areas of significant blood flow. With this in mind, the regulator 110 is adapted for being worn on a user 102 on or near their neck 104. The neck 104 may have a relatively irregular or uneven surface (e.g., is not a planar surface or smooth arcuate surface), but heat transfer is typically most effect with good solid/continuous and direct contact between the heat transfer components and the item being cooled/heated (i.e., the user's skin on their neck 104 and blood flowing near the skin surface).

To provide an enhanced heat transfer interface on the irregular/uneven surface of the neck 104, the regulator 110 includes a thermally conductive member 112 in the form of an elongated band or collar. Significantly, the thermally conductive member 112 is not formed of a metal or other rigid material. Instead, the member 112 is formed of a thermally conductive material that is not only flexible but is relatively soft and resilient (e.g., resiliently deformable), which may be termed “conformable” or “conforming” to the user's skin (or other nonplanar, nonsmooth, irregular/uneven surface). The thermally conductive member 112 may be molded and/or manufactured, for example but not as a limitation, with lamination, extrusion, or injection molding for mass production, and, in some cases, the Peltier unit may be embedded into molded material(s). By selecting a relatively soft and conformable material, the thermally conductive member 112 can be pressed against the user's neck 104 and make contact with much more cooling surface area on the skin of the neck 104, as compared to a rigid heat transfer element. The user's comfort is also significantly enhanced as there are no pressure points.

In this embodiment 110, a strap or band 116 is provided that extends from a both sides of a housing 122 over the member 112 to support the member 112 on the neck 104 and to apply a relatively uniform force/pressure upon the member 112 to urge it against the neck 104 to conform with the user's skin texture (e.g., to mold to the uneven surface of the neck). The strap or band 116 may include elastic components as is well-known in the clothing industry such that it is a one-size-fits-all regulator 110 or, it may include a clasp 117 such as a Velcro or similar clasp, that allows the size of the band/strap 116 to be adjusted, whereby the user 102 may control the tightness band 116 on the neck 104 (and, therefore, an amount of conforming pressure/force applied to the member 112, which may modify the percentage of contact surface area achieved, e.g., achieve 50 percent contact area when the band 116 is worn loosely versus 90 up to nearly 100 percent when worn tightly).

Generally, the thermally conductive member 112 may be formed of any material (or materials) that are flexible and conformable, so that they can take the shape of a user's body where cooling is desired, and that are also thermally conductive. Typically, the member 112 will be formed of a thickness (such as 0.1 to 0.375 inches or more) of a non-metallic material. In one embodiment, the thermally conductive material for member 112 is chosen to be a thermally conductive elastomer while in other cases thermally conductive polymers (or plastics) are used that provide the desired conforming characteristics. In this document, these groups of materials may be termed “thermally conductive and conformable materials.” In one exemplary implementation, the member 112 is formed from a conductive elastomer such as the CoolPoly® E or D-series elastomers available from Cool Polymers, Inc. (e.g., CoolPoly®g E8103 or the like) and are thermoplastic elastomers that have thermal conductivity from 1.0 W/mK to 15 W/mK (e.g., 5 to 75 times the thermal conductivity of conventional elastomers). These conductive elastomers may have a hardness of about Shore A 38 (e.g., similar to a soft eraser) or somewhat harder and can be molded to 3-dimensional shaped objects such as member 112 with conventional injection molding equipment. In other embodiments, thermally conductive plastics (or polymers) may be used such as the CoolPoly® E or D series plastics that have thermal conductivity in the range of 1.0 W/mK to 100 W/mK. Of course, other manufacturer's/distributer's thermally conductive material may be used to practice the invention such as, but not limited to, elastomeric thermal interface materials available from The Bergquist Company such as their Sil-Pad thermally conductive insulator products/materials, which provide desirable levels of thermal conductivity as well as being non-rigid and conformable to irregular surfaces (such as the a user's skin on their neck, head, back, wrist, knee, and the like). Also, the Peltier unit (e.g., module 120) may be injection molded into thermally conductive material.

As shown, the thermally conductive member 112 has a length that allows it to extend continuously about ⅓ to ⅔ about the user's neck 104, but, in other embodiments, a shorter or longer member 112 may be utilized in the regulator 110 (e.g., a length ranging from a 2 to 12 inches for partial coverage of the neck up to 18 inches or more for full coverage). The material used for the member 112 also typically may be stretched to conform to the user's neck 104 and/or to move with the user 102 during use (e.g., allow some movement of the user 102, which may be important when applied to joints or moving portions of the body such as the wrist, the knee, and the neck 104). The member 112 also has a height that may be varied to practice the invention, with the length and height being selected to provide a desired surface area for contacting and interfacing with the skin of the user 102, while a thickness (e.g., 0.1 to 0.25 inches or more) may be chosen to provide adequate amounts of thermally conductive material to conform to the skin's topography and also provide an adequate heat transfer pathway to remove (or deliver) heat. The thermally conductive member 112 acts to transfer heat away from the neck 104 in cooling operations of the regulator 110 and to transfer heat to the neck 104 in heating operations of the regulator.

To control the temperature of the thermally conductive member 102, the personal temperature regulator 110 includes a thermoelectric module 120. The thermoelectric module 120, as is explained in more detail below, is designed to use the Peltier effect to remove or add heat to the member 112. Briefly, the module 120 includes a thermoelectric or Peltier device that is powered by a low voltage and low current power source (such as a rechargeable battery) and creates a heat flux that allows it to draw heat away from (or transfer heat to) the abutting thermally conductive member 112 depending upon the direction of the current flow (e.g., by changing the bias of the polarity to switch from cooling to heating modes of operation). As shown, the module 120 includes a housing 122, such as a molded plastic body supporting the Peltier device and placing its heat transfer surface against the member 112, supporting a power source such as a battery and control components, and also supporting a heat sink cooled by fan 124 with air flow inlets and outlets (e.g., sidewalls with louvers or vents to allow air to be drawn into the housing 122 and openings to expel air from the fan 124). Although not shown in FIGS. 1 and 2, the regulator 110 may include an On/Off switch to control when the Peltier device and fan 124 are operated, and, further, a switch may also be provided to place the Peltier device into cooling or into heating operating mode (e.g., with the control components operating to change the flow polarity of the applied voltage to the differing materials of the Peltier device).

Due to the flexibility and shape-conforming nature of the thermally conductive material used in the regulators of the invention, the personal temperature regulators may be designed for positioning against a variety of locations on a person's or user's body and are not limited to use on the neck 104 as shown in FIGS. 1 and 2. For example, FIG. 3 illustrates another embodiment of a personal temperature regulator 310 that is adapted for use by the user 102 as a wristband worn on the wrist 306. The wrist 306 is a location where blood flow near the skin's surface is significant, and cooling of the blood flow can be achieved to regulate core body temperature of the user 102 through use of the regulator 310. As with the neck 104, the wrist 306 of the user 102 (or any other person) is not a smooth, regular surface but, instead, each person's wrist differs in size, shape, and the topography or surface of the skin. It will be understood that the cooling concepts taught herein may be applied to a wide variety of product designs to make use of the flexibility and conforming nature of the thermally conductive material, and these products may include both rigid and soft/squishy portions such as a computer mouse that may have a rigid base and housing portions but also a soft portion formed from thermally conductive material on an upper or side surface for mating with the irregular topography of a user's hand/finger(s) to cool their hand. A safety helmet provides another example of a product with strong and rigid components (e.g., the outer shell of the helmet) and with softer portions including a thermally conductive material element that may be provided in or attached to the helmet band to contact the wearer's head (such as on their forehead or the like) or provided on nearly any interior surface (e.g., when the helmet it a full head device such as worn by race car drivers, firefighters, and so on).

To provide enhanced heat transfer surface area/contact, the regulator 310 includes a thermally conductive member 312 that is formed of a flexible, conformable material that is thermally conductive (e.g., has an elongated body formed of an elastomeric material or conformable plastic/polymeric material with desirable thermal conductivity such as greater than about 1.0 W/mK or, in some cases greater than about 5 W/mK up to 15 W/m or more). The member 312 may have a length to allow it to extend completely about a typical user's wrist 306 but may include a gap to allow it to be applied to all wrists without compression along its length (e.g., stretching may be preferred to achieve a heat transfer contact or interface with the wrist 306). A wristband or strap 316 is provided that extends from the housing 322 to at least partially cover the member 312 and, when tightened (due to engaging a clasp (not shown) or due to elastic materials/components), to act to apply a conforming pressure/force upon the member 312 to urge it against the skin of the wrist 306 and, preferably, to conform to the shape of the wrist 306 to achieve a desired amount of direct contact (e.g., a heat transfer surface area or interface that is made up of at least about 50 percent of the surface area (or more than 75 percent in some embodiments and more than about 85 percent in others) of the contact surface or side of the member 312 facing the wrist 306). As with regulator 110, the regulator 310 includes a thermoelectric module 320 with a housing 322 for containing the Peltier device, a power source, control components (as appropriate), a heat sink contacting the Peltier device, and a fan 324 for drawing air into the housing 322 and over the heat sink prior to being exhausted from the housing 322.

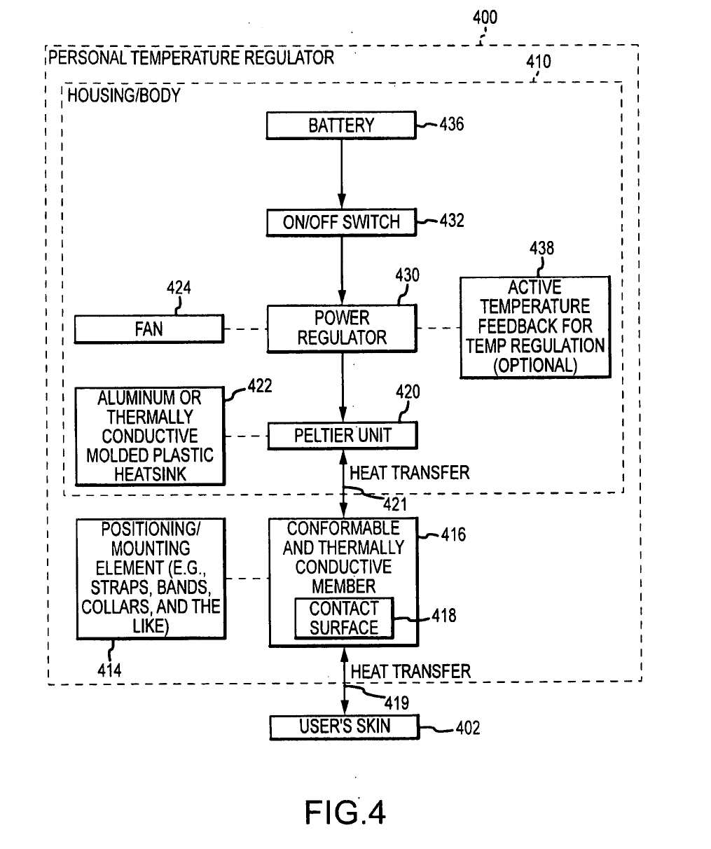

FIG. 4 illustrates a functional block diagram of a personal temperature regulator 400, which may be used to implement the regulators of FIGS. 1-3 and 5-12. The regulator 400 includes a housing or body 410 for supporting a number of regulator components including a battery 436 (e.g., a rechargeable or non-rechargeable battery such as a conventional rechargeable 9 Volt battery or the like). In some embodiments, the battery is replaced by a wired arrangement in which the regulator 400 is powered by a power source outside the housing 410 such as a battery pack worn by a user in another location on the body (e.g., on the waist or the like) or a wall or other plug-in socket. In other cases, charging is provided by solar devices, by piezo-electric charging devices, or by other charging components, which may be worn by the user (e.g., on the user's head in headgear or the like).

The battery 436 is the power source for providing electrical current to a number of regulator components including a fan 424 and a Peltier unit 420, and a power regulator 430 may be provided for regulating current transmitted to the fan 424 and to the Peltier unit 420. An On/Off switch 432 that is accessible by a user is provided on or in the housing 410 and is used to selectively provide power to the regulator 430 from the battery 436. Additional controls 438 may be provided to control operation of the regulator 400 such as active temperature feedback for temperature regulation and devices for changing the bias polarity of the power provided to the Peltier unit 420 to change the mode of operation between cooling and heating. Temperature regulation by components 438 may include providing a sensor(s) for determining the temperature of the thermally conductive member 416 (or the user's skin near such member 416), and, in response, attempting to operate the Peltier unit 420 to maintain the thermally conductive member 416 within a particular range (e.g., a range found to be comfortable to a user (and which may be adjustable by the user in some embodiments) such as 45 to 110 degrees or the like).

A thermoelectric cooling/heating device (or Peltier unit) 420 is provided in the regulator to transfer heat from or to a comformable and thermally conductive member 416. A heat sink 422 is provided in the housing 410 and placed with a surface in heat transfer contact with the Peltier unit 420. A fan 424 (such as a small fan often used in the computer and electronics industry for their relatively high capacity, low noise, and low weight) is provided to force air to flow over the heat sink surfaces to remove heat (or provide heat) to the heat sink 422. The heat sink 422 may be a finned design common for cooling electronics components such as CPUs and the like, and the heat sink 422 may be formed of a metal (such as aluminum to limit the weight of the sink or other thermally conductive material (e.g., a thermally conductive plastic such as an injection molded polymer)).

The Peltier unit 420 may take numerous forms that are commercially available or that is specifically designed for a particular regulator 410. In the cooling setting, the unit 420 may be thought of as a thermoelectric cooling device that uses the Peltier effect to create a heat flux between the junction of two different types of materials. It is typically designed as a solid-state active heat pump that transfers heat from one side of the device to the other side against the temperature gradient (i.e., from hot to cold) with the consumption of electrical energy provided by battery 436. In cooling mode of operation, the cold side of the Peltier unit 420 is placed against a side of the thermally conductive member 416 (i.e., the side that is opposite the skin-contacting side or heat transfer side/surface of the member 416). A typical Peltier unit 420 may be operable with low voltages (such as at a voltage provided by a conventional 9 V battery via regulator 430 such as 5 volts or the like) and may achieve large temperature differences proportional or based upon ambient temperatures, with some embodiments using Peltier units 420 that can be operated to cool the thermally conductive member 416 to a temperature of up to 40° F. or more different than ambient (e.g., 40° F. cooler than ambient in cooling mode and 40° F. higher than ambient in heating mode).

The Peltier unit 420 is positioned such that one layer of the junctioned, differing materials is exposed or extends out of the housing 410 to provide a heat transfer surface or interface. In FIG. 4, heat transfer 421 is shown to occur between the Peltier unit 420 and the thermally conductive member 416. Typically, one side of the thermally conductive member 416 is placed in abutting and solid (no air gap) contact with the exposed or protruding portion of the Peltier unit 420 to facilitate the heat transfer 421, e.g., with a surface area on this side at least as large as the exposed/protruding portion of the Peltier unit 420 positioned adjacent the Peltier unit 420. The comformable and thermally conductive member 416 may be formed of a thermally conductive elastomer or plastic as discussed above, and a positioning or mounting element 414 is used to allow the member 416 and the housing 410 to be worn by a user.

Specifically, the positioning/mounting element may include straps, bands, collars, and the like attached to or containing the housing 410 and being at least partially in contact with an exterior surface of the thermally conductive member 416 (e.g., in contact with the side of the member 416 that abuts the Peltier unit 420). When worn by a user (e.g., when strapped onto a wrist, knee, neck, torso, or the like), the mounting element 414 acts to urge the thermally conductive member 416 against the user's skin 402 such that a contact surface 418 on the side opposite that abutting the Peltier unit 420 is in solid contact (no or few air gaps) with the user's skin 402 to facilitate heat transfer 419. Particularly, the contact surface 418 is a relatively large portion (e.g., 50 to 90 percent or more) of the surface area on the user-facing/skin-contacting side of the member 416, and solid or proper heat transfer contact (pathways) is achieved as the conformable material of the member 416 is deformed to match the irregular surface and/or shape of the user's skin 402 (e.g., to become recessed to receive protruding skin covering bones or to protrude so as to extend into recessed portions of the skin).

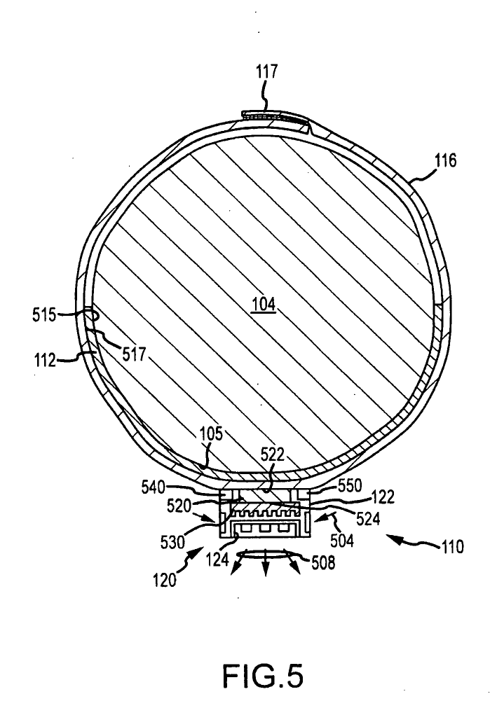

FIG. 5 illustrates a sectional view of a personal temperature regulator 110 attached to or worn on a user's neck 104. The regulator 110 includes a thermally conductive member 112 that is flexible and conformable (e.g., formed of a relatively soft plastic/polymer and/or of an elastomeric material) with a first side 515 placed in abutting or heat transfer contact with the skin 105 of the user's neck 104. A second side 517 of the thermally conductive member 112 is positioned to abut or contact a strap or band (e.g., positioning/mounting element) 116, which is used to hold the member 112 and thermoelectric module 120 in position and to apply a force against surface 517 of member 112 to force side 515 of member to conform to skin 105 of neck 104. As shown (with some amount of exaggeration), the neck 104 is a relative irregular cylindrical shape, and this results in the skin 105 having an irregular surface topography (e.g., not planar or smooth but instead with high and low points). The conforming nature of the member 112, though, allows the side 515 to conform and mold to the shape of the neck 104 or to the skin 105 in the contact portion of the neck 104, and, in some embodiments, a heat transfer junction is formed with over 50 percent of the surface area of the skin-contacting side 515 of the thermally conductive member 112 being in solid contact (e.g., no air gaps) with the skin 105 to provide a heat transfer pathway to and away from the skin 105. A clasp 117 may be used to connect the two ends of the strap 116 to allow the user to size the regulator 110 to their neck 104 and/or adjust the amount of pressure used to press the member 112 against the skin 105 (which may alter the fraction of the surface 515 in contact with the skin 105).

The side 517 of the thermally conductive member 112 is also in heat transfer or abutting contact with a side/surface 522 of the Peltier unit 520, which is contained in the housing 122 of module 120. The opposite side 524 of the Peltier unit 520 is mated to a heat sink 530 (such as with thermal grease or the like) so as to provide a heat transfer pathway away from the Peltier unit 520 during cooling operations (and to the unit 520 during heating operations). A fan 124 is positioned in the housing 122 and draws air 504 into the housing 122 such that it flows through the fins of the heat sink 530, and then the fan 124 forces the hotter/cooler air 508 out of the housing 122. The housing 122 may also contain a power source 540 such as a battery and control components 550 such as a power regulator to provide current at a particular voltage to the Peltier unit 520 and fan 124 and such as temperature sensing/regulating devices and/or an on/off switch.

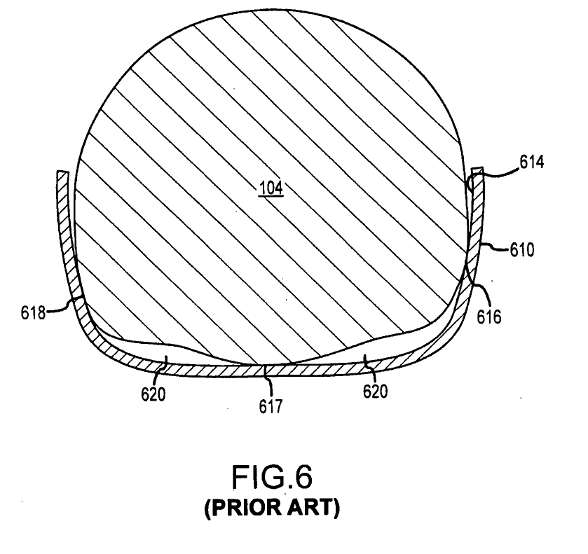

FIG. 6 is provided to illustrate that the conformable and flexible thermally conductive member 112 of FIG. 5 provides a significant advantage over a rigid thermal conductor. As shown, a thermal conductor such as a metallic band or partial collar 610 is shown as it may be worn on a user's neck 104. The collar 610 is rigid or only partially flexible such that its inner or contact surface 614 does not conform to the skin of the user's neck 104 (i.e., generally retains its original shape or cross-sectional topography, which is shown as a smooth arcuate shape). To be worn or retained on the neck 104, the collar 610 may be sized such that the user has to expand it a small amount and then place it onto their neck 104. However, the surface 614 does not conform to the irregular shape of the neck 104, but it instead contacts the skin of the neck 104 only at a small or limited number of contact points shown at 616, 617, 618 that typically will correspond to the high spots, ridges, corners, and the like of the neck 104 (or other body portion being cooled/heated). This only provides points (or lines) of heat transfer contact or interface, and only a relatively small amount of the surface 614 is in solid or heat transfer contact with the neck 104 (such as less than about 30 percent and, more typically, less than 10 percent), with the thermally insulating air gaps 620 being created between the neck 104 and the rigid conductor 610 next to these contact points 616, 617, 618. The effectiveness of a heat transfer device is greatly impacted by the amount of direct contact or heat transfer surface area available, and the arrangement shown in FIG. 6 provides a poor proportion of heat transfer surface area when compared with the personal temperature regulators designed in accordance with the invention (e.g., see the conforming thermally conductive member 112 of FIG. 5).

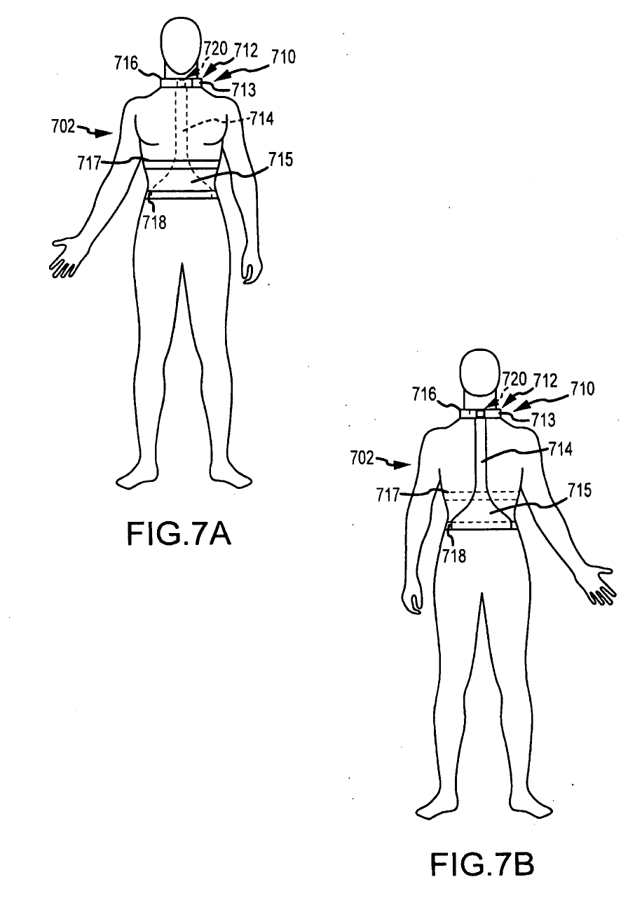

FIGS. 7A and 7B illustrate an embodiment of a personal temperature regulator 710 as it may appear when worn on a human body 702. As discussed earlier, there are a number of key or useful spots where cooling (or heating) may be used to more dramatically effect or better control core body temperature of the body 702, and these may coincide with areas where veins/arteries are near the skin (e.g., areas of near surface blood flow) such that the blood temperature can be controlled to affect a desired core body temperature. For example, it may be desirable to provide cooling at or near the neck, on the head, along the spine, in the lower back, on the wrists, inner arm, or underarm, on the backside of the or inner portion of the knee, and the like. With this in mind, the regulator 710 is adapted to place thermally conductive material near the neck, the spine, and the lower back of the user's body 702.

To this end, the personal temperature regulator 710 includes a thermally conductive member/assembly 712 that has a neck band 713 extending at least part way around the circumference of the neck of body 702. Also, the assembly 712 includes an elongated back or spinal band 714 extending from the neck band 713, which may be a band/strip of thermally conductive material with a thickness and length similar to that in the neck band 713 but with a width that is often greater than used on the neck (e.g., the neck band 713 may be 1 to 2 inches in width while the spinal band 714 may be 2 to 4 inches in width). Also, the length of the band 714 may be selected for expected heights of body 702 (or size of torsos of body 702) such as shorter for smaller torsos and longer for longer torsos (e.g., for taller users). The assembly 712 further includes a lower back member or pad 715 that extends from the spinal member 714 and is shaped (e.g., generally triangular as shown or other useful shapes such as circular, rectangular, and the like) to suit the recessed surface of the lower back.

To allow the user to wear the thermally conductive member assembly 712, the regulator 710 includes a strap or collar 716 for positioning the neck band 713 and two or more straps/bands (or other components such as a vest or the like) 717, 718 for positioning the spinal band 714 and the lower back pad 715. The straps/bands 716, 717, 718 act not only to retain the portions of the thermally conductive member assembly 712 but, preferably, to apply a force/pressure to the bands 713, 714 and pad 715 causing it to contact the skin of the body 702 and, in some cases, to conform to the topography of the skin to achieve a relatively large amount solid, heat transfer contact with the body 702 in these key areas of the neck, spine, and lower back. The regulator 710 also includes at least one thermoelectric module 720 in contact with one or more portions of the assembly 712 that acts to transfer heat away from (or to) the bands 713, 714 and pad 715. As shown, a single module 720 is provided in the regulator 710 and placed in contact with the neck band 713, but the neck band 713 is connected to the spinal bank 714, which, in turn, is connected to the lower back pad 715. Due to the location of the module 720, it may be desirable to provide less heat transfer surface area near the neck than in the lower back area (e.g., the portions of the assembly 720 proximate to the module 720 may be cooler (in cooling operations) than more distal portions).

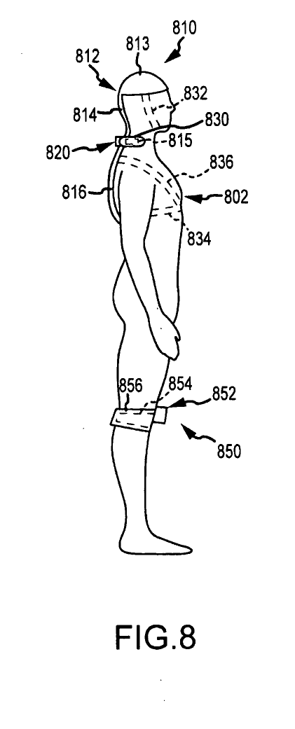

FIG. 8 illustrates another embodiment of a personal temperature regulator 810 for use in controlling the temperature of the user's body 802. The regulator 810 includes a thermally conductive material assembly 812 that includes a neck band 815 extending about a portion of the neck of the body 802, and a collar 830 is used to support the band 815 on the neck and to compress the band 815 against the skin. The neck band 815 of thermally conductive material is in heat conducting contact with a thermoelectric module 820 (e.g., a Peltier device with power source, heat sink, fan, and controls as discussed above) to remove heat from the neck band 815 in a cooling operating mode and to provide heat to the neck band 815 in a heating operating mode.

In some cases, it is desirable to cool a person's head to control their core body temperature. For example, a theme park or theater worker may wear a full body costume that covers the head, and it may be useful to cool their bead. Other workers such as welders, environmental remediation workers, hazardous material handlers, and so on may need to wear coveralls and hoods that cover their entire body including their head, and it may be useful to provide cooling directly to their head/scalp. To this end, the assembly 812 includes a skull cap 813 that is shaped and sized to at least partially cover the upper portion of the head of the user 802. A connector member 814 may be provided to provide a heat conduction pathway between the cap 813 and the thermoelectric module 820 (or the neck band 815, which may be in contact with the Peltier unit of module 820) to control the temperature of the thermally conductive material in the cap 813. The connector 814 may be applied to the back of the head to provide additional cooling/heating or may simply be used to transfer heat to and from the skull cap 813. Again, the cap 813 and neck band 815 (and, optionally, the connector 814) are formed of conformable, flexible material that is thermally conductive such as a thermally conductive elastomeric material, such that these components of assembly 812 better conform to the skin or other features of body 802 to achieve a large heat transfer surface area/interface between the assembly 812 and the body 802.

The assembly 812 further includes a back pad or portion 816 that may extend along the spine and provide a heat transfer interface with the lower back as shown in FIGS. 7A and 7B. To hold the assembly 812 in place and to apply a conforming pressure, a positioning/mounting assembly may be provided that as shown includes a number of straps such as a chin strap 832 to pull the skull cap 813 into contact with the top of the head and 2 to 3 or more straps 834, 836 that may extend over the shoulders and about the chest/torso to pull the back pad 816 against the skin of the back. The amount of pressure/force applied by straps 832, 834, 836 is typically chosen to cause the pad 816 and skull cap 813 to at least partially conform to the skin of these portions of the body 802 and achieve a desired fraction of heat transfer contact surface area (such as more than 50 percent of the available area of the cap 813 and pad 816). But, the amount of pressure may be balanced against comfort of the user 802, with the straps 832, 834, and/or 836 preferably being adjustable to suit the user 802.

The personal temperature regulator 810 may further include a pair of knee-mounted (or secondary) regulators 850 worn on the body 802 near or over each knee. The regulators 850 each include a thermoelectric module 852 with a Peltier unit for removing or providing heat when powered (such as by a battery in each module 852). An elastic band or adjustable strap 856 is used to support and position a thermally conductive band 854 that extends from the thermoelectric module 852. Preferably, the band 854 extends at least partially into the inner knee area where veins/arteries are near the surface of the skin to allow heat transfer to flowing blood in body 802. The band/strap 856 is used to apply a conforming pressure/force to the band 854 to cause the thermally conductive and conformable material of the band 854 contact the skin (which may have an irregular contour(s)) and be deformed to match the skin and achieve a large surface area for heat transfer in proportion to the surface area available in band 854 (again, up to 50 percent or more of the skin-facing side of the band 854 may be in solid or heat transfer contact with skin near the knee to provide a direct conductive pathway from heat away and/or to the skin (and blood flowing in arteries/veins near the skin)). The regulators 850 may be separately operable from each other and/or the module 820 or be operated in conjunction with these components to achieve a desired control over the core body temperature of the body 802.

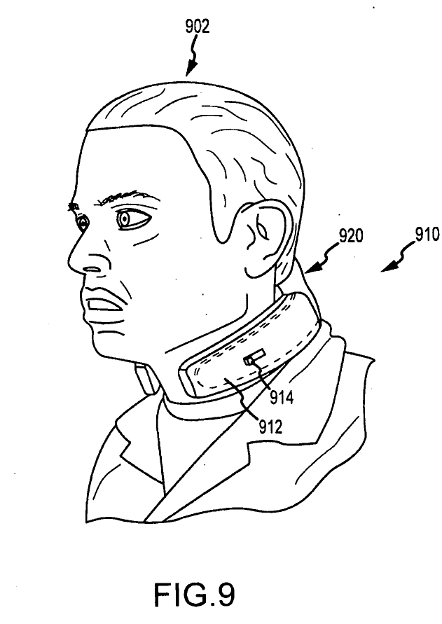

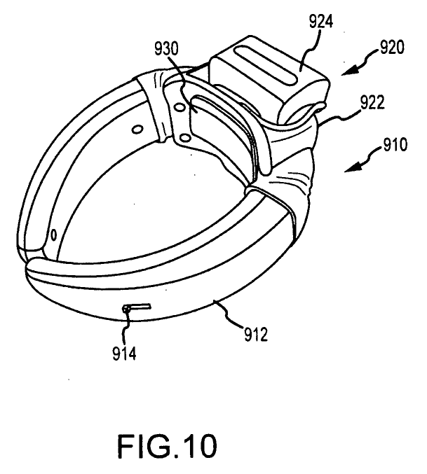

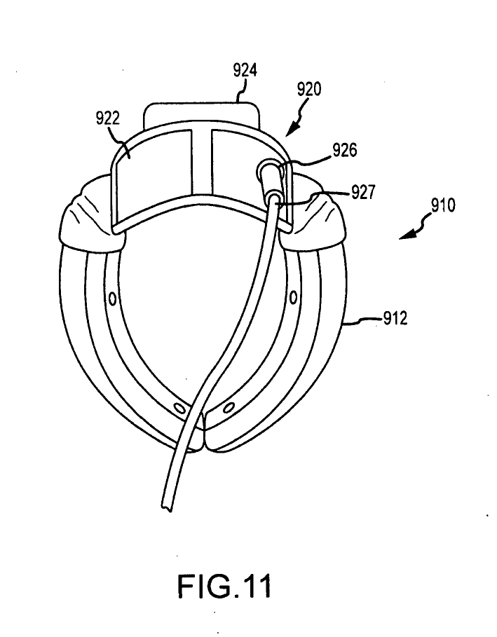

FIGS. 9-12 illustrate another embodiment of a personal temperature regulator 910 in use by a user 902. The regulator 902 is similar to the regulator 110 of FIGS. 1 and 2, and it is used to cool/heat the skin (and underlying flowing blood) on the neck of user 902 and, particularly, the skin near the back portion of the neck or near the spine (but a larger thermally conductive member may be utilized in some cases to extend about the neck of user 902). The regulator 910 includes a collar/body 912 that extends about the user's neck and is expandable/retractable to allow the collar 912 to be fit over the neck and then to retract toward the user's neck to hold the regulator in place on the user 902. The collar 912 may be formed, at least in part, of moldable plastic with the expandable/retractable feature provided by the elastic character of the plastic and/or with pivots/hinges in the corners between the sides of collar 912 and thermoelectric module housing 922 (see, for example, FIG. 10). In one embodiment, the collar 912 is formed using co-injection molded plastics with non-thermally conductive plastics like acrylonitrile butadiene styrene (ABS) or polystyrene (PS) with sections that are thermally conductive such as Sil-Pad™ materials or the like. Such a design may create areas that act as temperature conductors as well as areas that act as insulators. An On/Off switch 914 may be provided in the regulator 910 and positioned on collar 912 for ready access and operation by user 902 to selectively provide power to the thermoelectric module 920 (and, thus, turn cooling/heating on/off as needed).

The personal temperature regulator 910 further includes a thermoelectric module 920 linked to the collar 912. The module 920 includes a housing 922 that supports, in this case, an external power source 924 (e.g., a battery or the like with or without a power regulator or the power regulator may be provided in housing 922). The power source 924 may be a low voltage source (such as to power a low voltage Peltier unit (e.g., about 5 Volts or less in some cases)), and a power cord 927 may be plugged in at power/recharging receptacle 926 in housing 922. As shown in FIG. 10, the regulator 910 includes a thermally conductive member 930 that may include a sheet or layer of thermally conductive elastomer (or polymer, in some cases) that is selected for its ability to be molded or deformed to mate with an irregular surface (as typically of the human body including the back of the neck of a user 902) and for its ability to conduct heat (e.g., a thermal conductivity of 1 to 15 W/mK or more). In this embodiment, the thermally conductive member 930 is provided with a rectangular-shaped contact surface (e.g., a rectangle with a height of about 0.5 to 3 inches and a width of 2 to 6 inches) but other shapes and sizes may be utilized in the regulator 910. In use, the thermally conductive member 930 is pressed against the skin of the back of user's neck, by the configuration of the collar 912 and housing 922, and conforms (at least in part) to this skin texture.

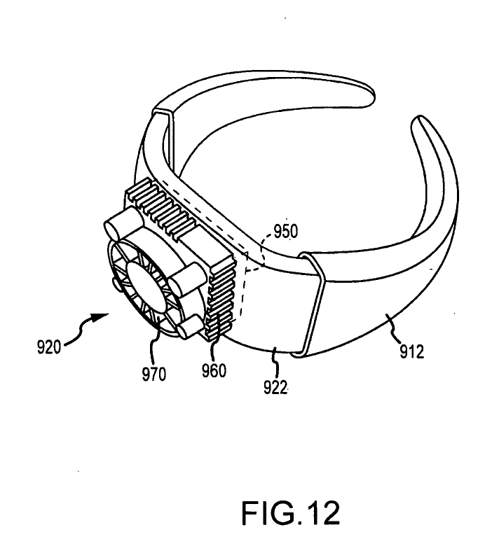

As shown with reference to FIG. 12, with a portion of the housing 922 removed, the thermoelectric module 920 includes a Peltier unit or device 950 that is positioned in housing 922 such that one of its surfaces/material layers contacts the thermally conductive member 930 (e.g., to provide a heat transfer pathway from the member 930 to the Peltier unit 950 to remove heat during cooling and add heat during heating operations of regulator 910). A heat sink 960 is placed in the housing 922 to have its base plate abut a side or surface of the Peltier unit 950 opposite the thermally conductive member 930, and it is formed of thermally conductive material and includes fins to increase the available surface area for transferring heat to air flowing through the housing 922. Air flow is provided by a fan 970 positioned near and/or on the fins of the heat sink 960. The fan 970 and the Peltier unit 950 are powered (typically with relatively low voltage) by electrical current supplied by power source 924.

The personal temperature regulators describe above may generally be described as making use of the relatively new and emerging technology of energy efficient Peltier junctions, e.g., Peltier devices that are operable at low voltages (such as less than about 9 Volts and the like). The regulators may include relatively small or miniaturized versions of Peltier junctions/devices to provide useful amounts of cooling such as cooling of a person's blood flowing near the surface of their skin. These small Peltier devices may be embedded into clothing, jewelry, and many other things (such as retrofitted into a ball cap, helmet, or the like) with one of the Peltier layer/materials (e.g., the cold element of the junction) placed in abutting contact with a thermally conductive element (e.g., a sheet of thermally conductive material such as an elastomer or flexible polymer or the like). The Peltier devices may also be provided in other products such as a computer mouse, a video game controller, and so on as the techniques described herein allow cooling (or other temperature regulatory properties) to be provided in nearly any product (e.g., any product that can be extruded, molded, and the like) to enhance user comfort. Embodiments of these regulators (or coolers or heaters) may be configured to run nearly silently (just a small amount of fan noise), efficiently and effectively (e.g., produce a 40° F. temperature differential relative to ambient temperatures at the surface/side of the thermally conductive member that is contacting a user's skin), and be powered with a rechargeable power source (e.g., a rechargeable battery) or even a renewable power source (e.g., a solar powered/charged battery or the like).

Although the invention has been described and illustrated with a certain degree of particularity, it is understood that the present disclosure has been made only by way of example, and that numerous changes in the combination and arrangement of parts can be resorted to by those skilled in the art without departing from the spirit and scope of the invention, as hereinafter claimed. The miniature or small packaging requirements of the personal temperature regulators in accordance of the invention allow the inventive coolers/heaters to be used in a variety of applications and form factors ranging from a bracelet to a neck band/collar to a cap to a knee pad to an assembly integrated into or worn under a worker's clothing (e.g., within a theme park worker's or other's customer or into a worker's protective coverall or suit or the like). The products incorporating the personal temperature regulators may typically be produced at relative low cost and are readily adaptable for use with casings, bodies, housings that may include themed components and/or branding. The power source may be remote from the thermoelectric module (or the housing containing the Peltier unit) such as in a battery pack worn by the user of the regulator with power and/or control wiring fed to the module housing.

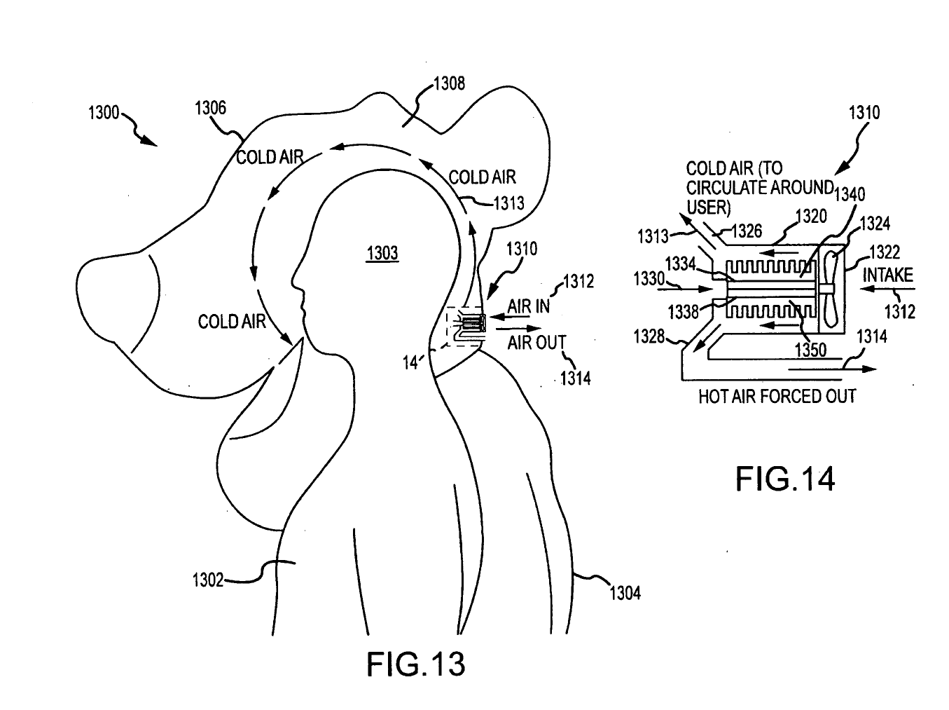

FIGS. 13 and 14 illustrate another embodiment of use of solid state cooling techniques to provide cooling to workers or other users that wear headgear such as a costume head, a protective helmet (e.g., a motorcycle helmet, a firefighter helmet, and so on), or a protective hood (e.g., as part of a hazardous material suit, anti-contamination clothing, and so on). A uniform, costume, suit, or protective clothing 1300 is shown that includes a personal temperature regulator 1310 in accordance with an embodiment of the invention to provide cooling within the suit 1300 (a costume in this example). The regulator 1310 may be used alone as shown or in combination with one of the other personal temperature regulators described herein. Briefly, the suit 1300 uses convection, forced-air cooling across thermally conductive heatsink(s) to cause cooled air to flow within one or more passageway within the suit 1300 (such as over the wearer's head or elsewhere) to cool the wearer or user of the suit 1300.

As shown, the suit 1300 includes a body portion 1304 and a head portion or headgear 1306 worn over a head 1303 of a wearer/user 1302. The suit 1300 includes a personal temperature regulator 1310 mounted (in this example but not as a limitation) on the headgear 1306 with an intake 1322 drawing air, air_{IN}, (shown at 1312) into the suit 1300 and expelling higher temperature air, air_{OUT}, (shown at 1314) after it has passed through a heat exchanger/heat sink to remove heat from the regulator 1310. The intake or incoming air 1312 is cooled within the regulator 1310 and directed as cooling or cold air 1313 into one or more passageways 1308 within the suit 1300, such as within the headgear/helmet 1306 as shown in FIG. 13 but other embodiments may direct the cooling air 1313 to other portions of the suit 1300. In the illustrated embodiment, the cooling air 1313 flows near the wearer's head 1303 and is at a temperature below the temperature of the incoming air 1312 (such as up to 10 to 20° F. or more cooler), which may be useful when the suit 1300 is worn in higher temperature environments (e.g., when the incoming air is about 80° F. or higher).

As shown in FIG. 14, the personal temperature regulator 1310 includes a housing 1320 with an air intake 1322 at one end (or one surface), and the regulator 1310 would be mounted or positioned upon the headgear 1306 such that the intake 1322 would be open to the environment to be able to draw in air 1312. Near the intake 1322, a fan 1324 is provided in the regulator 1310 to draw the air 1312 into the housing 1320. The regulator 1310 further includes a thermoelectric device or module (such as a Peltier unit) 1330 within the housing 1320 with a cool side/portion 1334 and a hot side/portion 1338. A power source (not shown) also typically would be included in the regulator 1310 to provide a driving power for the module 1330 (e.g., to cool the surface 1334). A pair of heatsinks 1340, 1350 is mounted on or placed in heat conducting contact with the surfaces/sides 1334, 1338 of the thermoelectric device 1330.

The housing 1320 is configured such that outside or intake air 1312 is caused to flow over heatsink 1340 to cause its temperature to drop to produce cold or cooling air 1313 that is directed to a cooling vent/outlet 1326 and into the interior passageway 1308 of the suit 1300. The cooling air 1313 may pass over the user's head 1303 and be expelled through one or more vents (not shown) in the headgear 1306 or body portion 1304 of the suit 1300. To remove heat from the thermoelectric module 1330, the housing 1320 is configured to direct a portion of the intake air 1312 over the second heatsink 1350, which abuts or is in heat conducting contact with the hot side of the thermoelectric device 1330, and a hot air outlet/vent 1328 is provided in the housing 1320 to expel the air 1314 after it has removed heat from the heatsink 1350 (and thermoelectric module 1330).

In some embodiments, ducting is attached to the regulator 1310 to provide the inlet air (or other gas such as oxygen-rich gas or the like) 1312. For example, the “head” 1308 may be a hood, a safety work helmet, another type of safety helmet (such as automobile or motorcycle helmet), and the like in addition to a costume or similar head as shown in FIG. 13. The ducting may be have one or two sources of supply air 1312 such that the incoming air may be cooling air or heating air. The ducting may also include valves or other devices to allow the source to be switched such that the flow of air 1313 may be either cold air as shown or warm air. Also, operation of the regulator 1310 may, in some cases, be reversed to cool or heat the incoming air 1312, as discussed in more detail above.

The concepts described herein are not limited strictly to personal temperature regulation or cooling/heating a person, as the concepts would also be applicable with or without modification to temperature regulation of objects and/or spaces (such as a room or the interior of a vehicle). In an automotive example or implementation, the air ducting techniques shown, for example, in FIGS. 13 and 14 may be used to cool or heat a vehicle such as while it is standing in a hot or cold environment. In such an implementation of a vehicle, the temperature regulator (or manifold 1310) may be provided within a dashboard, along or in a vehicle roof, and/or in/near the back window or dash. On a hot day, cold/cooler air may be circulated throughout the vehicle or over particular trouble spots (such as the driver's seat, the steering wheel, and so on). On a cold day, higher temperature air may be directed into a vehicle or to particular spots (such as to the driver's seat, to frosted windows, to the engine compartment, and so on). The regulator may be battery operated, may be powered with solar cells, or using other power sources.

In use, the vehicle (or space) temperature regulator would allow a user to leave their car or vehicle in the Sun for hours with the Sun powering the temperature regulator to draw outside air into the vehicle and to cool this air prior to directing it via its outlet manifold (and/or additional fans) into the vehicle to lower the temperature of the vehicle. When the vehicle user returned, the vehicle would be comfortable to enter or at least less hot (e.g., provide a reduction of 10 to 30 degrees or the like). More than one temperature regulator may be used to achieve additional cooling. When such cooling is combined with Sun blocking windows and other temperature control devices (such as solar-powered exhaust fans), the temperature of the vehicle may be significantly reduced. In turn, this may reduce the amount of energy required to cool the vehicle during initial use, and air conditioning use is typically often at its peak upon initially starting a vehicle (e.g., without the vehicle temperature regulator of the invention, the internal temperature may need to be reduced 40 to 50 degrees or more in hotter climates). Likewise, the vehicle temperature regulator may be used in winter months to maintain a warmer interior temperature of a vehicle that is left outside (such as when solar or battery powered) or garage (such as when battery powered or solar charged-battery powered).

We claim:

1. An apparatus for regulating a user's body temperature, comprising:
a thermoelectric module comprising a Peltier unit providing a heat transfer surface at a temperature differing from ambient temperature; and
a thermally conductive member with a first side abutting the heat transfer surface of the Peltier unit and a second side opposite the first side for contacting an area of skin of a user's body, wherein the thermally conductive member comprises a material with physical properties whereby the second side conforms to a topography of the skin in the contact area on the user's body.

2. The apparatus of claim 1, wherein the thermally conductive member is flexible and conformable and the material comprises a thermally conductive elastomeric material.

3. The apparatus of claim 1, wherein the second side comprises a contact surface and wherein at least about 50 percent of the contact surface mates with the skin to provide a conductive heat transfer pathway from the skin to the thermally conductive member.

4. The apparatus of claim 3, wherein at least about 75 percent of the contact surface mates with the skin to provide the conductive heat transfer pathway from the skin to the thermally conductive member.

5. The apparatus of claim 1, wherein the material has a thermal conductivity greater than about 1 W/mK.

6. The apparatus of claim 1, wherein the material has a hardness of less than about Shore A 40 and wherein the temperature of the heat transfer surface differs from ambient temperature by at least about 30° F.

7. The apparatus of claim 1, wherein thermoelectric module further comprises a housing supporting the Peltier unit, wherein the thermally conductive member comprises an elongated body extending outward from the housing, and wherein the apparatus further comprises a positioning element attached to the housing and extending over a portion of the first side of the thermally conductive member, the positioning element applying a compressive force upon the thermally conductive member when worn on the user's body urging the second side against the skin in the contact area.

8. The apparatus of claim 7, wherein the thermoelectric module further comprises a low voltage power source operating at a voltage of less than about 9 Volts and wherein the Peltier unit operates to create the temperature at the heat transfer surface in response to electrical current provided by the low voltage power source.

9. A personal temperature regulator, comprising:
a thermoelectric device powered by current from a low voltage power source to provide a heat transfer surface with a temperature differential relative to ambient; and
a thermally conductive member with a body having a first side in heat transfer contact with the heat transfer surface of the thermoelectric device and a second side for positioning in abutting contact with a user wearing the personal temperature regulator, wherein the body comprises a material conformable to the user.

10. The personal temperature regulator of claim 9, wherein the material of the body comprises a thermally conductive elastomer.

11. The personal temperature regulator of claim 10, wherein the thermally conductive elastomer has a thermal conductivity of greater than about 1 W/mK.

12. The personal temperature regulator of claim 9, wherein the thermoelectric device comprises a Peltier unit, the temperature differential is at least about 30 F, and the low voltage power source comprises a battery with a 9 Volt or lower voltage rating to provide the current to the Peltier unit to achieve the temperature differential at the heat transfer surface.

13. The personal temperature regulator of claim 9, wherein the body comprises a substantially planar sheet of the conformable material, the personal temperature regulator further comprising a positioning element for applying a compressive force on the first side of the body when worn by the user to urge the second side of the body against the user's skin, whereby at least about 50 percent of the second side is in abutting contact with the user's skin.

14. The personal temperature regulator of claim 13, wherein the body and positioning element are adapted for facilitating positioning the second side of the body proximate at least one human body feature selected from the group consisting of: neck, head, wrist, knee, and back.

15. An apparatus for cooling a user by lowering a user's core body temperature, comprising:
a skin-contacting member comprising a planar body of a thermally conductive elastomeric material;
a thermoelectric module comprising a housing and a Peltier device positioned within the housing in heat transfer contact with a portion of the planar body of the skin-contacting member; and
a positioning element for applying a force on the planar body, when the apparatus is worn by the user, to urge a contact surface of the planar body against an area of the user's skin that has a non-planar topography, whereby the thermally conductive elastomeric material at least partially conforms to the non-planar topography of the user's skin.

16. The apparatus of claim 15, wherein the area of the user's skin is proximate to at least one of the user's neck, head, wrist, back, or knee.

17. The apparatus of claim 16, wherein the positioning element comprises an article of clothing wearable by the user and wherein when the positioning element is worn by the user the skin-contacting member directly contacts the user's skin to provide a conductive pathway for heat to be transferred between the skin-contacting member and the user's skin.

18. The apparatus of claim 15, wherein the thermally conductive elastomeric material conforms to the non-planar topography of the user's skin to create a solid contact between the planar body and at least about 50 percent of the area of the user's skin.

19. The apparatus of claim 15, wherein the Peltier device comprises a heat transfer surface for contacting the portion of the planar body of the skin-contacting member, the heat transfer surface having a temperature at least about 30° F. less than an ambient temperature when an electrical current is provided from a power source electrically connected to the Peltier device.

20. The assembly of claim 19, wherein the power source is a low voltage power source providing the electrical current at a potential of less than about 9 Volts.
